(19) United States
(12) Patent Application Publication
(10) Pub. No.: US 2009/0304794 A1
(43) Pub. Date: Dec. 10, 2009

(54) CONTROLLED RELEASE FORMULATIONS OF PRAMIPEXOLE

(75) Inventors: Michael L. Vieira, Gaithersburg, MD (US); Padmanabh P. Bhatt, Rockville, MD (US); Austin B. Huang, N. Potomac, MD (US)

Correspondence Address: FOLEY AND LARDNER LLP;SUITE 500, 3000 K STREET NW, WASHINGTON, DC, 20007, US

(73) Assignee: Supernus Pharmaceuticals, Inc.

(21) Appl. No.: 12/478,979

(22) Filed: Jun. 5, 2009

Related U.S. Application Data
(60) Provisional application No. 61/129,175, filed on Jun. 9, 2008.

(51) Int. Cl. A61K 9/24; A61K 31/428; A61P 25/00

(52) U.S. Cl. 424/473; 514/367

(57) ABSTRACT

A controlled release formulation of pramipexole for once-a-day administration to a mammalian subject, which formulation releases pramipexole along a pre-determined release profile, is provided.

### CROSS-REFERENCE TO RELATED APPLICATION

This application claims priority from U.S. Provisional Application No. 61/129,175, filed Jun. 9, 2008, the entire contents of which are incorporated herein by reference.

### BACKGROUND

This invention is directed to oral controlled release dosage forms of pramipexole, which is a nonergot dopamine D2/D3 receptor agonist (NEDA). Pramipexole is indicated for the treatment of the signs and symptoms of idiopathic Parkinson's disease as well as for the treatment of moderate to severe primary restless leg syndrome (RLS). Pramipexole is commercially available under the tradename Mirapex® (pramipexole dihydrochloride) in an immediate release (IR) tablet at the following does: 0.125 mg, 0.25 mg, 0.5 mg, 1 mg and 1.5 mg. RLS patients are prescribed one dose 2-3 hours before bedtime, while Parkinson's patients are prescribed to take a tablet three times a day (TID).

Mirapex® exhibits linear pharmacokinetics over its clinical dose range and is rapidly absorbed following oral administration reaching Cₘₐₓ in about 2 hours. The drug has a large volume of distribution (approximately 500 L), is only moderately bound to plasma proteins (15%), and distributes into red blood cells (erythrocyte to plasma ratio 2:1). The absolute bioavailability (BA) of pramipexole is greater than 90%, and the terminal elimination half-life of pramipexole is about 8 hours in healthy volunteers and about 12 hours in elderly volunteers.

There is also a gender difference in the clearance of the drug: females have a 30% lower clearance rate than males. Also, because the drug is primarily excreted in the urine unmetabolized, dose adjustments must be made in patients having renal impairment.

Pramipexole dosage in Parkinson's disease is titrated based on the patient's response starting at 0.125 mg tid for a total daily dose intake of 0.375 mg. Since adverse events such as dyskinesia, hallucinations, orthostatic hypotension, somnolence and dry mouth are observed when the dose of pramipexole is increased, dose titrations are targeted to achieve maximum therapeutic effect while balancing the adverse events.

There is a need for a dosage form that delivers the needed amount of pramipexole once-daily thereby improving patient compliance. Furthermore, there is a need for a once-daily controlled release dosage form, which form can potentially provide a better therapeutic profile while minimizing unwanted side effects.

### SUMMARY OF THE INVENTION

The invention presents a controlled release formulation of pramipexole for once-a-day administration. The total amount of pramipexole in the formulation may vary from 0.375 mg to 9 mg.

In one embodiment of the invention, the controlled release formulation is an osmotic formulation comprising a therapeutically effective amount of pramipexole, an osmotic agent and a semipermeable membrane.

In another embodiment of the invention, a controlled release formulation comprises a release modifying polymer selected from a release delaying polymer selected from a group consisting of Eudragit FS 30 D (poly(methyl acrylate-co-methyl methacrylate-co-methacrylic acid)), Eudragit L and S (poly(methacrylic acid-co-methyl methacrylate))hydroxypropyl methylcellulose phthalate, hydroxypropyl methylcellulose acetate succinate, cellulose acetate phthalate, cellulose acetate trimelliate, polyvinyl acetate phthalate, shellac, and zein; an extended release polymer selected from a group consisting of cellulose acetate, cellulose acetate butyrate, cellulose acetate propionate and derivatives thereof, cellulose acylate, ethylcellulose, polyvinyl acetate, Eudragit NE 30 D poly(ethyl acrylate-co-methyl methacrylate), Eudragit RS and RL poly(ethyl acrylate-co-methyl methacrylate-cotrimethylammonioethyl methacrylate chloride)ammonio methacrylate copolymer Type B NF and ethyl acrylate methyl methacrylate copolymer, or combinations thereof.

In yet another embodiment of the invention, the controlled release formulation comprises at least one extended release component and at least one immediate release component.

Controlled release formulations of the current invention may comprise more than one extended release component, each characterized by its own release profile, or a combination of at least one extended release component and a delayed release component.

The current invention additionally provides dosage forms for once-a-day administration of the controlled release formulation, as well as the method of treatment of Parkinson's disease, restless leg syndrome, and other central nervous system disorders using this formulation.

### BRIEF DESCRIPTION OF DRAWINGS

FIG. 1 provides a summary of the adverse events reported for each noted formulation.

FIG. 2 provides a summary of the gastrointestinal adverse events reported for each noted formulation.

FIG. 3 provides a summary of the nervous system adverse events reported for each noted formulation.

FIG. 4 provides simulated steady state plasma profiles of osmotic formulations of pramipexole.

FIG. 5 provides simulated steady state plasma profiles of osmotic formulations of pramipexole with an additional IR component.

### DEFINITIONS

For the purposes of this invention, the term “pramipexole” includes pramipexole or any pharmaceutically acceptable salt thereof, as well as any crystalline and non-crystalline forms, and any polymorph(s).

An “immediate release formulation” refers to a formulation that releases greater than or equal to 80% of the pharmaceutical agent in less than or equal to about 1 hour.

“Extended release” is defined herein as release of a pharmaceutical agent in a continuous manner over a prolonged period of time. When used in reference to pharmaceutical ingredients per se that confer “extended release,” the term is used synonymously with “release controlling.”

By “prolonged period of time” is meant a continuous period of time of greater than 1 hour, preferably, greater than 4 hours, more preferably, greater than 8 hours, more preferably greater than 12 hours, more preferably still, greater than 16 hours up to more than 24 hours.

As used herein, unless otherwise noted, “rate of release” or “release rate” of a drug refers to the quantity of drug released from a dosage form per unit time, e.g. milligrams of drug released per hour (mg/hr) or a percentage of a total drug dose released per hour. Drug release rates for dosage forms are typically measured as an in vitro rate of drug release, i.e. a quantity of drug released from the dosage form per unit time measured under appropriate conditions and in a suitable fluid in the laboratory. The time at which a specified percentage of the drug within a dosage form has been released from said dosage form is referred to as the “Tx” value, where “x” is the percent of drug that has been released.

The release rates referred to herein are determined by placing the dosage form to be tested in an appropriate dissolution media bath. Aliquots of the medium, collected at pre-set intervals, are then injected into a chromatographic system fitted with an appropriate detector to quantify the amounts of drug released during the testing intervals.

“C” denotes the concentration of the drug typically in blood plasma, or serum, of a subject, and is generally expressed as mass per unit volume, for example nanograms per milliliter (ng/ml). For convenience, this concentration may be referred to herein as “drug plasma concentration,” “plasma drug concentration” or “plasma concentration” which is intended to be inclusive of a drug concentration measured in any appropriate body fluid or tissue. The plasma drug concentration at any time following drug administration is referenced as Ctime, as in C9 hr or C4 hr, etc.

The maximum plasma drug concentration during the dosing period is referenced as Cmax, while Cmin refers to the minimum blood plasma drug concentration at the end of a dosing interval; and Cave refers to an average concentration during the dosing interval.

The “percent of fluctuation” for a dosing period is defined as a quotient (Cmax−Cmin)/Cave*100%.

Persons of skill in the art will appreciate that plasma drug concentrations obtained in individual subjects will vary due to inter-patient variability in the many parameters affecting drug absorption, distribution, metabolism and excretion. For this reason, unless otherwise indicated, when a plasma drug concentration is listed, the value listed is the calculated mean value based on values obtained from a group of subjects tested.

The term “bioavailability” refers to an extent to which, and sometimes the rate at which, the active moiety (drug or metabolite) enters the systemic circulation, thereby gaining access to the site of action.

“AUC” is the area under the plasma concentration-time curve and is considered to be the most reliable measure of bioavailability. The AUC is directly proportional to the total amount of unchanged drug that reaches the systemic circulation.

Side effect is defined herein as any undesirable secondary, usually adverse, effect of a drug.

For the purposes of this application, two formulations are given in the “equivalent amount” if they produce an AUC within 80% to 125% of each other for the same period of time.

Throughout this application, “administered tid” means that ⅓ of the total daily dose of the active agent is administered every 8 hours.

Unless otherwise specified, “a” or “an” means “one or more”.

### DETAILED DESCRIPTION OF THE INVENTION

Controlled release formulations of the current invention are designed in such a way that pramipexole is released from the formulation along a pre-determined release profile. In one embodiment, a once-a-day administration of the formulation of the current invention results in the bioavailability that is equivalent to that produced by the equivalent amount of pramipexole administered as an immediate release formulation TID.

In another embodiment of the invention, the pre-determined release profile of the inventive formulation is such that a maximum steady state plasma concentration (Cmax) of pramipexole is not higher than the maximum plasma concentration produced by the equivalent amount of pramipexole administered as an immediate release formulation TID, and a minimum steady state plasma concentration (Cmin) is not lower than 75% of the minimum plasma concentration produced by the equivalent amount of pramipexole administered as an equivalent immediate release formulation TID.

In yet another embodiment, the profile is such that the degree of fluctuation is in the range of from 50% to 125% of the degree of fluctuation produced by the equivalent amount of pramipexole administered as an immediate release formulation TID.

The current invention comprises a formulation of pramipexole such that at least 80% of the active ingredient is released in a time period of from 12 to 24 hours, and preferably, in a time period of from 12 to 14 hours. Alternatively, the formulation may be designed in a way that at least 80% of the active ingredient is released in the time period of from 16 to 18 hours. In a further embodiment, at least 80% of the active ingredient is released in the time period of from 20 to 24 hours.

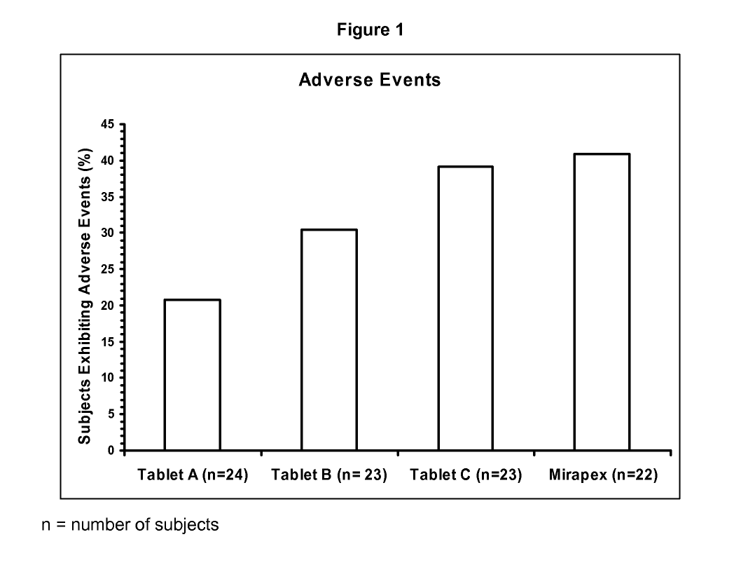

Formulations of the current invention have a decreased level of undesirable side effects as compared to the equivalent amount of pramipexole administered as an immediate release formulation TID. The side effects that are potentially reduced include dyskinesia, nausea, dizziness, hallucinations, orthostatic hypotension, somnolence, headache and dry mouth, among others (FIG. 1). In a clinical trial in 24 healthy adult subjects, adverse events (AEs) were recorded in 21% of the subjects receiving Tablet A and in 30% of subjects receiving Tablet B versus 41 % of subjects receiving Mirapex®. The compositions of the tablets are described in Example 1.

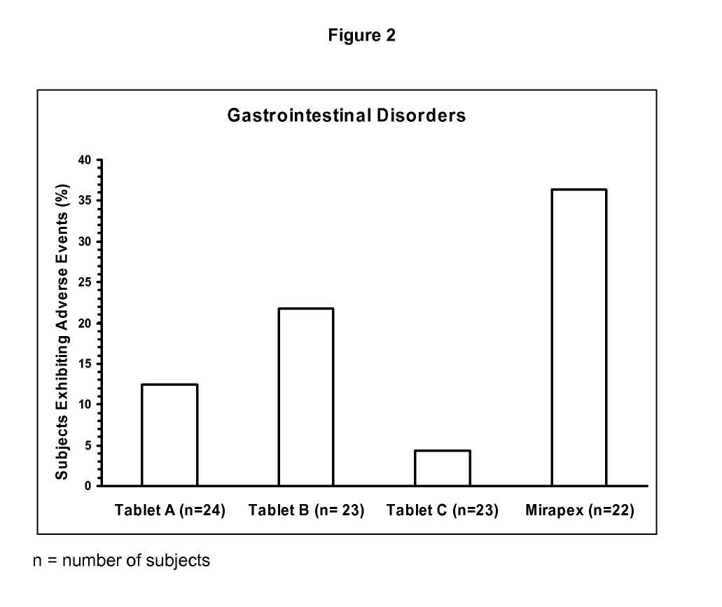

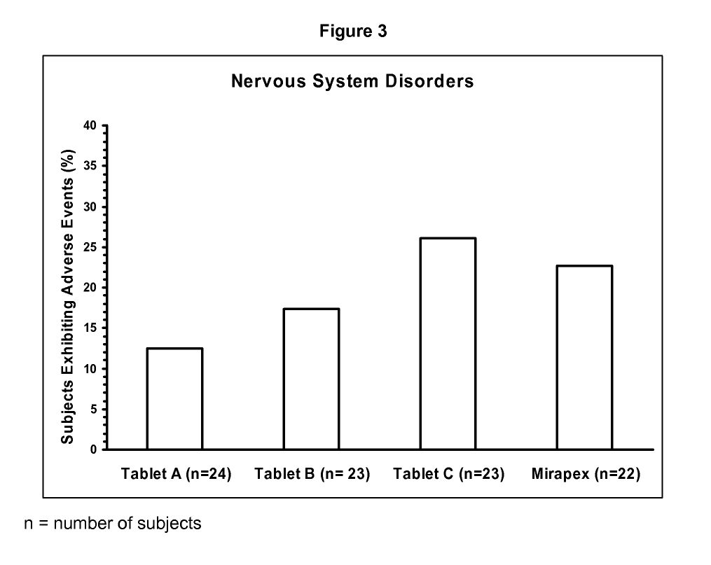

The most frequent AEs, classified by system organ class, involved the gastrointestinal and the nervous systems (FIGS. 2 and 3, respectively). The gastrointestinal adverse events reported for Tablet A, Tablet B and Tablet C were lower than those reported for Mirapex® ((4%-22%, and 36%, respectively). The nervous system adverse events reported for Tablet A (13%) and Tablet B (17%) were also lower than that for Mirapex® (23%); however, AEs reported for Tablet C (26%) were comparable to those reported for Mirapex®.

In one embodiment of the invention, the pre-determined release profile is achieved by incorporating pramipexole into an osmotic formulation comprising pramipexole, a non-swellable osmotic agent, and a semipermeable membrane, wherein the amount of the osmotic agent is from about 5 to 90 weight percent. The osmotic agents are thought to promote the flux of water through the semipermeable membrane resulting in solubilization of the water-soluble components of the core tablet.

In another embodiment, the pre-determined profile is achieved by applying a small amount (up to 10% of the total dose) of pramipexole as an immediate release formulation over an osmotic formulation described herein, thus forming an immediate release layer. The IR layer may be applied by any drug coating method known in the art.

Without putting any limitations thereon, the osmotic agent may be selected from a range of non-swellable, water-soluble agents, including but not limited to sugars, non-reducing sugars in particular, such as mannitol, xylitol, sorbitol, isomalt, trehelose, maltilol, sucrose, and erythritol; inorganic salts such as sodium chloride, potassium chloride, sodium phosphate, and potassium phosphate; and organic acids and salts, such as ascorbic acid, aspartame, malic acid, tartaric acid, citric acid, sodium ascorbate, sodium citrate, potassium citrate, sodium bicarbonate, sodium carbonate, and sodium acetate.

The formulations of the present invention may be presented in a dosage form selected from a tablet, a pill, a capsule, a caplet, a troche, a sachet, a cachet, a pouch, powder or sprinkles. In one embodiment, the formulation is presented in the form of an osmotic tablet dosage form comprising a core tablet; a release controlling, semipermeable membrane that is applied to the core tablet; and an orifice, drilled mechanically or by laser through the semipermeable membrane, which orifice provides an exit port for solubilized components of the core tablet.

The core tablet is a compressed tablet formulation comprising (a) pramipexole, (b) a non-swellable osmotic agent (e.g. mannitol and/or isomalt), a binder selected from povidone, starch, gelatin, maltodextrin, methylcellulose, hydroxypropyl cellulose, hydroxypropyl methylcellulose, carboxymethylcellulose, sucrose solution, dextrose solution, acacia, tragacanth and locust bean gum; and (c) a lubricant, such as sodium stearyl fumarate and the metallic stearates among others (magnesium stearate).

Additionally, wetting and solubilizing agents such as sodium docusate, sodium lauryl sulfate, polyethylene glycol, lecithin, poloxamer, the polysorbates, the polyoxethylene ethers and the sorbitan esters; diluents such as microcrystalline cellulose, dicalcium phosphate, calcium sulfate, cellulose, starch, and talc; disintegrants such as crosslinked sodium carboxymethylcellulose, sodium starch glycolate and crospovidone; buffering agents and/or pH modulating agents, such as aluminum hydroxide, ammonium bicarbonate, ammonium carbonate, ammonium phosphate, arginine, calcium acetate, calcium ascorbate, magnesium acetate, magnesium carbonate, potassium acetate, potassium bicarbonate, potassium carbonate, potassium phosphate dibasic, potassium sodium tartrate, potassium citrate, sodium citrate, sodium phosphate monobasic, sodium phosphate dibasic, sodium phosphate tribasic, sodium acetate, sodium bicarbonate, sodium ascorbate, sodium carbonate, fumaric acid, malic acid, tartaric acid, ascorbic acid, aspartic acid, alginic acid, glutamic acid, sorbic acid, and succinic acid; and glidants such as talc, starch and colloidal silicon dioxide may be added to the core tablet formulation.

Briefly, the core tablet may be processed as follows: the core tablet formulation components (with the exception of the lubricant) are processed into granules using a fluid bed processor and water as the granulating fluid. The granulation is dried in the fluid bed, passed through an 18 mesh screen to remove agglomerates and then blended with the lubricant (magnesium stearate) using a powder blender. The resultant granulation is then compressed into tablets on a rotary tablet press. Alternatively the core tablet may be produced by dry blending/direct compression techniques known in the art.

The core of the tablets of the current invention may be a single-layer core or a bilayer core comprising more than one active ingredient containing layer, wherein each layer is characterized by its own release profile.

The semipermeable membrane may be applied to the core tablets using a pan coating technique. The semipermeable membrane formulation comprises at least one release controlling polymer and at least one plasticizer. The formulation optionally may include membrane permeability enhancers (e.g., water soluble excipients) to further modulate the flux of water into the core tablet. Release controlling polymers suitable for forming a semipermeable membrane include cellulose acetate, cellulose acetate butyrate, cellulose acetate propionate and derivatives thereof, cellulose acylate and ethylcellulose, among others.

In embodiments where an IR layer is present, a solution of the drug and a suitable binder (such as hypromellose, povidone) may be applied to the exterior of the osmotic tablet using a pan coating technique.

Optionally, a protective coating layer may be applied on top of the semipermeable membrane or on top of the additional IR layer. Polymers suitable for forming such coatings include: hydroxypropyl methylcellulose including the commercially available coating systems (e.g., Opadry), polyvinyl alcohol and aminoalkyl methacrylate copolymer.

The invention is further illustrated by, though in no way limited to, the following examples.

### EXAMPLES

### Example 1

The following table provides non-limiting examples for three formulations of Pramipexole XR (i.e., Tablet A, Tablet B and Tablet C).

TABLE 1

| | | Tablet A | | Tablet B | | Tablet C | |
|---|---|---|---|---|---|---|---|
| | | Quantity | | Quantity | | Quantity | |
| Ingredient | Function | (mg) | % (w/w) | (mg) | % (w/w) | (mg) | % (w/w) |
| Pramipexole | Drug | 0.75 | 0.36 | 0.75 | 0.36 | 0.75 | 0.36 |
| Dihydrochloride | | | | | | | |
| Monohydrate^{a} | | | | | | | |
| Mannitol, USP | Osmotic | 103.80 | 49.90 | 103.80 | 49.71 | 103.80 | 49.24 |
| | agent | | | | | | |
| Isomalt, NF | Osmotic | 86.78 | 41.72 | 86.78 | 41.56 | 86.78 | 41.17 |
| | agent | | | | | | |
| Povidone, USP | Binder | 6.67 | 3.21 | 6.67 | 3.19 | 6.67 | 3.16 |
| Magnesium | Lubricant | 2.00 | 0.96 | 2.00 | 0.96 | 2.00 | 0.95 |
| Stearate, NF^{b} | | | | | | | |
| Cellulose Acetate, | Release | 6.40 | 3.08 | 7.04 | 3.37 | 8.64 | 4.10 |
| NF | Controlling | | | | | | |
| | polymer | | | | | | |
| Triethyl Citrate, | Plasticizer | 1.60 | 0.77 | 1.76 | 0.84 | 2.16 | 1.02 |
| NF | | | | | | | |
| | Total | 208.0 | 100 | 208.8 | 100 | 210.8 | 100 |

^{a}The drug products are formulated to provide the pramipexole dose strength consistent with the commercially available immediate release tablet formulations, Mirapex ® (pramipexole dihydrochloride tablets). The “label dose” strength of Mirapex ® tablets is based on the drug substance form pramipexole dihydrochloride monohydrate.
^{b}Vegetable origin

### Example 2

The pharmacokinetic profiles of the three extended-release formulations of pramipexole of Table 1 (single dose 0.75 mg) were evaluated in a 4-way, crossover pilot study in healthy adult subjects using Mirapex® as the comparator (0.25 mg every eight hours for a total dose of 0.75 mg per day).

The pharmacokinetic parameters for the formulations of Example 1 are as follows:

TABLE 2

| | Tablet A | Tablet B | Tablet C | Mirapex |
|---|---|---|---|---|
| Total Dose (mg) | 0.75 | 0.75 | 0.75 | 0.75 |
| Cₘₐₓ (pg/mL) | 688.4 | 613.4 | 549.4 | 669.5 |
| tₘₐₓ (hr) | 16.0 | 16.0 | 16.0 | 18.0 |
| t_{1/2} (hr) | 9.9 | 10.0 | 9.7 | 9.4 |
| AUC₀₋₂₄ (pg hr/mL) | 11300.4 | 9951.8 | 8172.7 | 11792.7 |
| AUCₗₐₛₜ (pg hr/mL) | 18762.8 | 18421.4 | 16646.0 | 17636.7 |
| AUC_{inf} (pg hr/mL) | 18840.3 | 18712.8 | 16907.6 | 18045.6 |
| Rel BA (AUC₀₋₂₄) | 0.96 | 0.84 | 0.69 | NA |
| Rel BA (AUCₗₐₛₜ) | 1.06 | 1.04 | 0.94 | NA |
| Rel BA (AUC_{inf}) | 1.04 | 1.04 | 0.94 | NA |

### Example 3

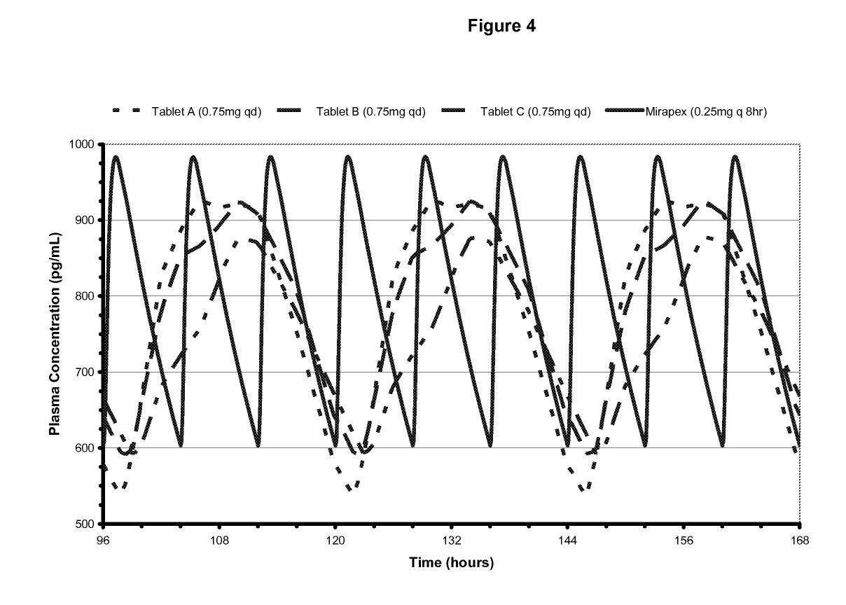

The pharmacokinetic parameters of Table 2 were used as a basis for the in silico steady state plasma profile simulations for Tablets A, B, and C. The results of the simulation are represented in Table 3 and in FIG. 4. WinNonlin® version 5.0.1 and 5.2 (Pharsight Corporation, Mountain View, Calif. 94041) and GastroPlus™ version 5.3 and 6.0 (Simulations Plus, Inc., West Lancaster, Calif. 93534) were used to perform in silico simulations.

TABLE 3
Steady State Simulations

| | Tablet A | Tablet B | Tablet C | Mirapex |
|---|---|---|---|---|
| Total Dose (mg) | 0.75 | 0.75 | 0.75 | 0.75 |
| Cmax (pg/mL) | 926 | 926.2 | 877.8 | 983.5 |
| Cmin (pg/mL) | 536.9 | 592.3 | 592.1 | 603.3 |
| Cave (pg/mL) | 785.3 | 791.5 | 745 | 797.1 |
| AUC144 hr-168 hr | 18847.3 | 18661.9 | 17880.3 | 19130 |
| Fluctuation (%) | 49.5 | 42.2 | 38.3 | 47.7 |
| Relative BA | 0.99 | 0.98 | 0.93 | 1.00 |
| AUC144 hr-168 hr | | | | |

### Example 4

IR component-containing Tablets A1, B1, and C1 were prepared from tablet formulations A, B, and C of Example 1 by coating a layer of pramipexole (0.075 mg) over Tablets A, B and C, respectively.

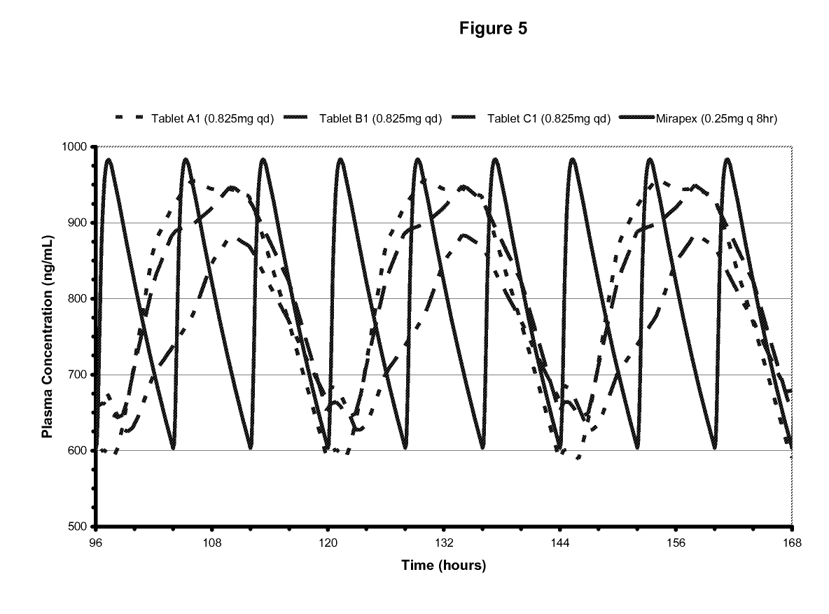

The pharmacokinetic parameters of Table 2 were used as a basis for in silico steady state plasma profile simulations for Tablets A1, B1 and C1. The results of the simulation are represented in Table 4 and in FIG. 5.

WinNonlin® version 5.0.1 and 5.2 (Pharsight Corporation, Mountain View, Calif. 94041) and GastroPlus™ version 5.3 and 6.0 (Simulations Plus, Inc., West Lancaster, Calif. 93534) were used to perform the in silico simulations.

TABLE 4
Steady State Simulations

| | Tablet | Tablet | Tablet | |
|---|---|---|---|---|
| | A1 | B1 | C1 | Mirapex |
| Total Dose (mg) | 0.825 | 0.825 | 0.825 | 0.75 |
| Cmax (pg/mL) | 957.2 | 949.5 | 885.2 | 983.5 |
| Cmin (pg/mL) | 589 | 644.7 | 625.8 | 603.3 |
| Cave (pg/mL) | 814.4 | 820.7 | 756.8 | 797.1 |
| AUC144 hr-168 hr | 19546.4 | 19697.5 | 18162.7 | 19130 |
| Fluctuation (%) | 45.2 | 37.1 | 34.3 | 47.7 |
| Dose Normalized | 0.93 | 0.94 | 0.86 | 1.00 |
| Relative BA | | | | |
| AUC144 hr-168 hr | | | | |
| Non-Dose | 1.02 | 1.03 | 0.95 | 1.00 |
| Normalized | | | | |
| Relative BA | | | | |
| AUC144 hr-168 hr | | | | |

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

1. A pharmaceutical formulation of pramipexole for once-a-day administration comprising a therapeutically effective amount of pramipexole, and an osmotic agent, wherein pramipexole is released from the formulation along a pre-determined release profile.

2. The formulation of claim 1, further comprising an extended release polymer.

3. The formulation of claim 1, wherein the osmotic agent is a non-reducing sugar.

4. The formulation of claim 3 wherein said non-reducing sugar is mannitol, xylitol, sorbitol, isomalt, trehelose, maltilol, sucrose, erythritol, or a combination thereof.

5. The formulation of claim 2 further comprising a semipermeable membrane comprising the extended release polymer.

6. The formulation of claim 5 wherein said extended release polymer comprises cellulose acetate, cellulose acetate butyrate, cellulose acetate propionate and derivatives thereof, cellulose acylate, ethylcellulose, and combinations thereof.

7. The formulation of claim 5 further comprising a layer of an immediate release pramipexole coated onto the semipermeable membrane.

8. The formulation of claim 1 further comprising a pharmaceutically acceptable excipient.

9. The formulation of claim 8 wherein the pharmaceutically acceptable excipient comprises binders, lubricants, plasticizers, glidants, diluents, wetting agents, solubilization agents, and combinations thereof.

10. The formulation of claim 2, wherein the extended release polymer is selected from the group consisting of cellulose acetate, cellulose acetate butyrate, cellulose acetate propionate and derivatives thereof, cellulose acylate, ethylcellulose, polyvinyl acetate, Eudragit NE 30 D poly(ethyl acrylate-co-methyl methacrylate), Eudragit RS and RL poly (ethyl acrylate-co-methyl methacrylate-cotrimethylammonioethyl methacrylate chloride).

11. The formulation of claim 1, wherein said formulation releases at least 80% of the pramipexole in the period of time from 12 to 24 hours.

12. The formulation of claim 11, wherein said formulation releases at least 80% of the pramipexole in the period of time from 12 to 14 hours.

13. The formulation of claim 11, wherein said formulation releases at least 80% of the pramipexole in the period of time from 16 to 18 hours.

14. The formulation of claim 11, wherein said formulation releases at least 80% of the pramipexole in the period of time from 20 to 24 hours.

15. The formulation of claim 1 in a dosage form selected from a tablet, a pill, a capsule, a caplet, a troche, a sachet, a cachet, a pouch, powder or sprinkles.

16. The formulation of claim 15, wherein said tablet is an osmotic tablet comprising a core and a semipermeable membrane.

17. The formulation of claim 16, wherein said core is a monolayer core or a bilayer core.

18. The formulation of claim 16, additionally comprising a layer of an immediate-release pramipexole coated onto the semipermeable membrane.

19. The formulation of claim 18, wherein said layer contains up to 10% of the total amount of pramipexole in the dosage form.

20. The formulation of claim 1, comprising at least one pramipexole-containing extended release component.

21. The formulation of claim 20, further comprising at least one immediate release component.

22. The formulation of claim 20, further comprising at least one delayed release component.

23. The formulation of claim 20, further comprising at least one immediate release component and at least one delayed release component.

24. A method of treating a subject suffering from a central nervous system disorder, comprising administering to the subject a once-daily pharmaceutical formulation of pramipexole comprising a therapeutically effective amount of pramipexole and an osmotic agent.

25. The method of claim 24, wherein the central nervous system disorder is Parkinson's disease, Restless Legs syndrome, or both.

26. The method of claim 24, wherein the administration lowers the incidence rate and severity of side effects as compared to an immediate release formulation of pramipexole.

27. The method of claim 26, wherein said side effects are gastrointestinal side effects and nervous system side effects.

28. The method of claim 27, wherein the incidence of gastrointestinal side effects is reduced by at least 20%.

29. The formulation of claim 1 for the treatment of a central nervous system disorder.
